(19) United States
(12) Patent Application Publication
(10) Pub. No.: US 2024/0148861 A1
(43) Pub. Date: May 9, 2024

(54) ENGINEERED VARICELLA ZOSTER VIRUS SHINGLES MRNA VACCINE

(71) Applicant: Vernagen, LLC, Tucker, GA (US)

(72) Inventors: Baek KIM, Atlanta, GA (US); Jack Yoon, Suwanee, GA (US); David Pak, Suwanee, GA (US)

(73) Assignee: Vernagen, LLC, Tucker, GA (US)

(21) Appl. No.: 18/504,503

(22) Filed: Nov. 8, 2023

Related U.S. Application Data
(60) Provisional application No. 63/423,759, filed on Nov. 8, 2022.

(51) Int. Cl. A61K 39/25; A61P 31/22

(52) U.S. Cl. CPC A61K 39/25; A61P 31/22; A61K 2039/53

(57) ABSTRACT

Provided herein are a shingles vaccine composition including a messenger ribonucleic acid (mRNA) including an open reading frame (ORF) encoding varicella Zoster virus (VZV) glycoprotein E (gE)(which is soluble VZV gE or full-length VZV gE) and a method of inducing immune response against Shingles by administering an effective amount of the Shingles vaccine composition to a subject in need thereof.

### CROSS REFERENCE TO RELATED APPLICATIONS

This application claims priority to U.S. Provisional Application No. 63/423,759 filed Nov. 8, 2022, the entire disclosure of which is incorporated herein by reference.

### INCORPORATION BY REFERENCE OF SEQUENCE LISTING

The content of the electronically submitted sequence listing, file name: A292636_sequence listing as filed; size: 25,789 bytes; and date of creation: Nov. 6, 2023, filed herewith, is incorporated herein by reference in its entirety.

### FIELD

Provided herein are a shingles vaccine composition including a messenger ribonucleic acid (mRNA) comprising an open reading frame (ORF) encoding varicella zoster virus (VZV) glycoprotein E (gE)(which is soluble VZV gE or full-length VZV gE), and a method of inducing immune response against Shingles by administering an effective amount of the Shingles vaccine composition to a subject in need thereof.

### BACKGROUND

Shingles, also known as zoster or herpes zoster, is a viral disease and caused by varicella zoster virus (VZV). At present, there is no approved shingles mRNA vaccine, and there has been a need for shingles mRNA vaccine.

### SUMMARY

The present disclosure provides a shingles vaccine composition comprising a messenger ribonucleic acid (mRNA) comprising an open reading frame (ORF) encoding varicella zoster virus (VZV) glycoprotein E (gE). In one embodiment, the VZV gE has an amino acid sequence of SEQ ID NO: 1 (soluble VZV gE). In another embodiment, the VZV gE has an amino acid sequence of SEQ ID NO: 2 (full-length VZV gE). In one embodiment, the ORF encoding VZV gE has a nucleotide sequence of SEQ ID NO: 3 (the ORF encoding soluble VZV gE). In another embodiment, the ORF encoding VZV gE has a nucleotide sequence of SEQ ID NO: 4 (the ORF encoding full-length VZV gE). In some embodiment, the mRNA comprising the ORF encoding VZV gE further comprises a 5′ untranslated region (UTR), a 3′ UTR, and a poly (A) tail so as to have the structure of 5′UTR-ORF encoding VZV gE-3′UTR-poly (A) tail, and the ORF encoding VZV gE has a nucleotide sequence of SEQ ID NO: 3 (the ORF encoding soluble VZV gE). In another embodiment, the mRNA comprising the ORF encoding VZV gE further comprises a 5′ untranslated region (UTR), a 3′ UTR, and a poly (A) tail so as to have the structure of 5′UTR-ORF encoding VZV gE-3′UTR-poly (A) tail, and the ORF encoding VZV gE has a nucleotide sequence of SEQ ID NO: 4 (the ORF encoding full-length VZV gE). In one embodiment, the poly (A) tail has a length of 50-250 nucleotides. In some embodiment, the mRNA having the structure of 5′UTR-ORF encoding VZV gE-3′UTR-poly (A) tail has a nucleotide sequence of SEQ ID NO: 5 (5′UTR-ORF encoding soluble VZV gE-3′UTR-poly (A) tail). In another embodiment, the mRNA having the structure of 5′UTR-ORF encoding VZV gE-3′UTR-poly (A) tail has a nucleotide sequence of SEQ ID NO: 6 (5′UTR-ORF encoding full-length VZV gE-3′UTR-poly (A) tail). In some embodiment, the mRNA having the structure of 5′UTR-ORF encoding VZV gE-3′UTR-poly (A) tail has a nucleotide sequence having at least 80% identity to SEQ ID NO: 5 (5′UTR-ORF encoding soluble VZV gE-3′UTR-poly (A) tail). In another embodiment, the mRNA having the structure of 5′UTR-ORF encoding VZV gE-3′UTR-poly (A) tail has a nucleotide sequence having at least 80% identity to SEQ ID NO: 6 (5′UTR-ORF encoding full-length VZV gE-3′UTR-poly (A) tail). In one embodiment, the shingles vaccine composition of the present disclosure further comprises a pharmaceutically acceptable carrier. In one embodiment, the pharmaceutically acceptable carrier is a lipid nanoparticle encapsulating the mRNA therein.

The present disclosure also provides a method of inducing immune response against shingles comprising administering an effective amount of the shingles vaccine composition of the present disclosure to a subject in need thereof.

### BRIEF DESCRIPTION OF THE DRAWINGS

FIG. 1 shows in vitro transcription for two VZV gE mRNAs.

FIG. 2 shows VZV gE protein levels in gE mRNA-transfected 293FT cells and culture media.

FIG. 3 describes the schedule of immunizations and sera collections.

FIG. 4 shows mouse anti-VZV-gE titer results.

FIG. 5 shows VZV neutralization results.

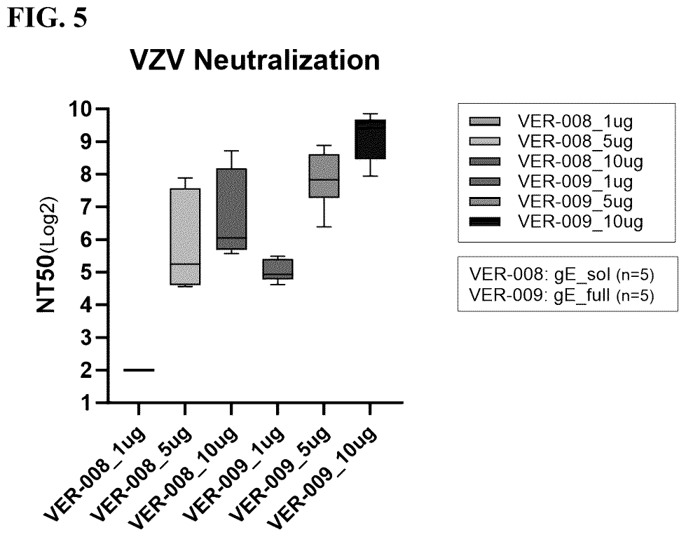

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this disclosure is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this disclosure belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc., and excludes any unrecited feature(s), element(s), method step(s), etc., except for ordinarily-associated impurities. The phrase “consisting essentially of” denotes the recited feature(s), element(s), method step(s), etc., and any additional feature(s), element(s), method step(s), etc., that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “shingles vaccine composition” refers to a substance used to stimulate the production of antibodies and provide immunity against shingles.

As used herein, the term “messenger ribonucleic acid (mRNA)” refers to a single-stranded molecule of RNA that corresponds to the genetic sequence of a gene, and is read by a ribosome in the process of synthesizing a protein.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodemosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

As used herein, the term “open reading frame (ORF)” refers to a nucleotide sequence between the start and stop codons.

As used herein, the term “an open reading frame (ORF) encoding” refers to the nucleotide coding sequence which encodes a polypeptide. The coding sequence can further include initiation and termination signals operably linked to regulatory elements including a promoter and polyadenylation signal capable of directing expression in the cells of an individual or mammal to which the nucleic acid is administered. The coding sequence can further include sequences that encode signal peptides.

As used herein, the term “T7 promoter” refers to a promoter derived from a bacteriophage T7.

As used herein, the term “5′ untranslated region (UTR)” refers to a region of an mRNA that is directly upstream (i.e., 5′) from the start codon (the first codon of an mRNA transcript translated by a ribosome) that does not encode a polypeptide.

As used herein, the term “3′ untranslated region (UTR)” refers to a region of an mRNA that is directly downstream (i.e., 3′) from the stop codon (i.e., the codon of an mRNA transcript that signals a termination of translation) that does not encode a polypeptide.

As used herein, the term “poly (A) tail” refers to a long stretch of adenine nucleotides added to the “tail” or 3′ end of the mRNA.

As used herein, the term “pharmaceutically acceptable carrier” refers to any substance or vehicle suitable for delivering a mRNA vaccine to a suitable in vivo or ex vivo site. Such a carrier can include, but is not limited to, an adjuvant, an excipient, a lipid particle, etc.

As used herein, the term “lipid nanoparticle” refers to a particle having at least one dimension on the order of nanometers (e.g., 1-1,000 nm). In some embodiments, lipid nanoparticles are included in a formulation that can be used to deliver a mRNA vaccine to a target site of interest (e.g., cell, tissue, organ, tumor, and the like). In some embodiments, the mRNA vaccine, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response. In some embodiments, the lipid nanoparticle has a mean diameter of 50-200 nm. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol. In some embodiments, the cationic lipid is selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

As used herein, the term “inducing immune response against shingles” refers to providing protective immunity and/or vaccinating a subject against shingles for prophylactic purposes, as well as causing a desired immune response or effect in a subject in need thereof against shingles, for therapeutic purposes. As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all.

An “effective amount” of the shingles vaccine composition (e.g. mRNA) is provided based, at least in part, on the target tissue, target cell type, means of administration, physical characteristics of the polynucleotide (e.g., size, and extent of modified nucleosides) and other components of the vaccine, and other determinants. In general, an effective amount of the shingles vaccine (e.g., mRNA) provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the RNA, e.g., mRNA, vaccine), increased protein translation from the polynucleotide, decreased nucleic acid degradation (as demonstrated, for example, by increased duration of protein translation from a modified polynucleotide), or altered antigen specific immune response of the host cell.

As used herein, the term “X % identity to SEQ ID NO: Y” or “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “nucleotide sequence having at least X % identity to SEQ ID NO: Y and encodes Z protein” means that the nucleotide sequence meets the two different requirements of having at least X % identity to SEQ ID NO: Y and encoding Z protein.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The terms “subject,” “patient,” “individual,” and the like are used interchangeably herein, and refer to any animal, any mammalian subject, or cells thereof whether in vitro or in situ, amenable to the methods described herein. In certain non-limiting embodiments, the patient, subject or individual is a human.

### DETAILED DESCRIPTION

1. The Shingles Vaccine Composition

The present disclosure provides a shingles vaccine composition comprising an open reading frame (ORF) encoding varicella zoster virus (VZV) glycoprotein E (gE).

In one embodiment, the VZV gE has an amino acid sequence of SEQ ID NO: 1 (soluble VZV gE). In another embodiment, the VZV gE has an amino acid sequence of SEQ ID NO: 2 (full-length VZV gE).

In one embodiment, the VZV gE has an amino acid sequence having at least 80% identity to SEQ ID NO: 1 (soluble VZV gE). In another embodiment, the VZV gE has an amino acid sequence having at least 85% identity to SEQ ID NO: 1 (soluble VZV gE). In some embodiment, the VZV gE has an amino acid sequence having at least 90% identity to SEQ ID NO: 1 (soluble VZV gE). In another embodiment, the VZV gE has an amino acid sequence having at least 95% identity to SEQ ID NO: 1 (soluble VZV gE). In one embodiment, the VZV gE has an amino acid sequence having at least 96% identity to SEQ ID NO: 1 (soluble VZV gE). In some embodiment, the VZV gE has an amino acid sequence having at least 97% identity to SEQ ID NO: 1 (soluble VZV gE). In another embodiment, the VZV gE has an amino acid sequence having at least 98% identity to SEQ ID NO: 1 (soluble VZV gE). In some embodiment, the VZV gE has an amino acid sequence having at least 99% identity to SEQ ID NO: 1 (soluble VZV gE).

In another embodiment, the VZV gE has an amino acid sequence having at least 80% identity to SEQ ID NO: 2 (full-length VZV gE). In some embodiment, the VZV gE has an amino acid sequence having at least 85% identity to SEQ ID NO: 2 (full-length VZV gE). In one embodiment, the VZV gE has an amino acid sequence having at least 90% identity to SEQ ID NO: 2 (full-length VZV gE). In some embodiment, the VZV gE has an amino acid sequence having at least 95% identity to SEQ ID NO: 2 (full-length VZV gE). In another embodiment, the VZV gE has an amino acid sequence having at least 96% identity to SEQ ID NO: 2 (full-length VZV gE). In some embodiment, the VZV gE has an amino acid sequence having at least 97% identity to SEQ ID NO: 2 (full-length VZV gE). In another embodiment, the VZV gE has an amino acid sequence having at least 98% identity to SEQ ID NO: 2 (full-length VZV gE). In some embodiment, the VZV gE has an amino acid sequence having at least 99% identity to SEQ ID NO: 2 (full-length VZV gE).

The present disclosure provides two different types of shingles vaccine compositions as follows.
Shingles vaccine composition (1): a shingles vaccine composition comprising a mRNA comprising an ORF encoding VZV gE (soluble)
Shingles vaccine composition (2): a shingles vaccine composition comprising a mRNA comprising an ORF encoding VZV gE (full-length)

In the above two types of the shingles vaccine compositions, the VZV gE (soluble) may have an amino acid sequence of SEQ ID NO: 1 (or an amino acid sequence having at least 80%, 85%, 90%, 95%, 96%, 97%, 98%, 99% or 99% identity to SEQ ID NO: 1). In another embodiment, the VZV gE (full-length) may have an amino acid sequence of SEQ ID NO: 2 (or an amino acid sequence having at least 80%, 85%, 90%, 95%, 96%, 97%, 98%, 99% or 99% identity to SEQ ID NO: 2).

In the above two types of the shingles vaccine compositions, the ORF encoding VZV gE (soluble) may have a nucleotide sequence of SEQ ID NO: 3 (or a nucleotide sequence having at least 80%, 85%, 90%, 95%, 96%, 97%, 98%, 99% or 99% identity to SEQ ID NO: 3). In another embodiment, the ORF encoding VZV gE (full-length) may have a nucleotide sequence of SEQ ID NO: 4 (or a nucleotide sequence having at least 80%, 85%, 90%, 95%, 96%, 97%, 98%, 99% or 99% identity to SEQ ID NO: 4).

In the shingles vaccine composition (1), the mRNA comprising the ORF encoding VZV gE (soluble) may further comprise a 5′ untranslated region (UTR), a 3′ UTR, and a poly (A) tail so as to have the structure of 5′UTR-ORF encoding VZV gE (soluble)-3′UTR-poly (A) tail, and the ORF encoding VZV gE (soluble) may have a nucleotide sequence of SEQ ID NO: 3 (or a nucleotide sequence having at least 80%, 85%, 90%, 95%, 96%, 97%, 98%, 99% or 99% identity to SEQ ID NO: 3).

In the shingles vaccine composition (2), the mRNA comprising the ORF encoding VZV gE (full-length) may further comprise a 5′ untranslated region (UTR), a 3′ UTR, and a poly (A) tail so as to have the structure of 5′UTR-ORF encoding VZV gE (full-length)-3′UTR-poly (A) tail, and the ORF encoding VZV gE (full-length) may have a nucleotide sequence of SEQ ID NO: 4 (or a nucleotide sequence having at least 80%, 85%, 90%, 95%, 96%, 97%, 98%, 99% or 99% identity to SEQ ID NO: 4).

In the shingles vaccine composition (1), the mRNA having the structure of 5′UTR-ORF encoding VZV gE (soluble)-3′UTR-poly (A) tail may have a nucleotide sequence of SEQ ID NO: 5 (or a nucleotide sequence having at least 80%, 85%, 90%, 95%, 96%, 97%, 98%, 99% or 99% identity to SEQ ID NO: 5).

In the shingles vaccine composition (2), the mRNA having the structure of 5′UTR-ORF encoding VZV gE (full-length)-3′UTR-poly (A) tail may have a nucleotide sequence of SEQ ID NO: 6 (or a nucleotide sequence having at least 80%, 85%, 90%, 95%, 96%, 97%, 98%, 99% or 99% identity to SEQ ID NO: 6).

In one embodiment, the poly (A) tail has a length of 50-250 nucleotides. In another embodiment, the poly (A) tail has a length of 100-200 nucleotides. In another embodiment, the poly (A) tail has a length of 110-150 nucleotides. In another embodiment, the poly (A) tail has a length of 115-125 nucleotides. In another embodiment, the poly (A) tail has a length of 116-124 nucleotides. In another embodiment, the poly (A) tail has a length of 117-123 nucleotides. In another embodiment, the poly (A) tail has a length of 118-122 nucleotides. In another embodiment, the poly (A) tail has a length of 119-122 nucleotides. In another embodiment, the poly (A) tail has a length of 115 nucleotides. In another embodiment, the poly (A) tail has a length of 116 nucleotides. In another embodiment, the poly (A) tail has a length of 117 nucleotides. In another embodiment, the poly (A) tail has a length of 118 nucleotides. In another embodiment, the poly (A) tail has a length of 119 nucleotides. In another embodiment, the poly (A) tail has a length of 120 nucleotides. In another embodiment, the poly (A) tail has a length of 121 nucleotides. In another embodiment, the poly (A) tail has a length of 122 nucleotides. In another embodiment, the poly (A) tail has a length of 123 nucleotides. In another embodiment, the poly (A) tail has a length of 124 nucleotides. In another embodiment, the poly (A) tail has a length of 125 nucleotides.

In one embodiment, the mRNA of the present disclosure may comprise at least one chemical modification selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In another embodiment, the chemical modification is in the 5-position of the uracil. In another embodiment, the chemical modification is a N1-methylpseudouridine. In another embodiments, the chemical modification is a N1-ethylpseudouridine.

In one embodiment, the shingles vaccine composition further comprises a pharmaceutically acceptable carrier. In another embodiment, the pharmaceutically acceptable carrier may include any substance or vehicle suitable for delivering a mRNA vaccine to a suitable in vivo or ex vivo site. Such a carrier can include, but is not limited to, an adjuvant, an excipient, a lipid particle, etc. The lipid nanoparticle may be a particle having at least one dimension on the order of nanometers (e.g., 1-1,000 nm). In some embodiments, lipid nanoparticles are included in a formulation that can be used to deliver a mRNA vaccine to a target site of interest (e.g., cell, tissue, organ, tumor, and the like). In some embodiments, the mRNA vaccine, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response. In some embodiments, the lipid nanoparticle has a mean diameter of 50-200 nm. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol. In some embodiments, the cationic lipid is selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

In one embodiment, the lipid nanoparticle comprises (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), (ii) a neutral lipid selected from DSPC, DPPC, POPC, DOPE and SM, (iii) a sterol, e.g., cholesterol, and (iv) a PEG-lipid, e.g., PEG-DMG or PEG-cDMA, in a molar ratio of about 20-60% cationic lipid:5-25% neutral lipid:25-55% sterol; 0.5-15% PEG-lipid.

In one embodiment, the lipid nanoparticle includes from about 25% to about 75% on a molar basis of a cationic lipid selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about to about 65%, from about 45 to about 65%, about 60%, about 57.5%, about 50% or about 40% on a molar basis.

In one embodiment, the lipid nanoparticle includes from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In some embodiments, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), and PEG-cDMA.

In one embodiment, the lipid nanoparticle includes 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In one embodiment, the lipid nanoparticle include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In one embodiment, the lipid nanoparticle includes 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In one embodiment, the lipid nanoparticle includes about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In one embodiment, the lipid nanoparticle includes about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In one embodiment, the lipid nanoparticle includes about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In one embodiment, the lipid nanoparticle includes about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In one embodiment, the shingles vaccine composition of the present disclosure may be delivered, localized and/or concentrated in a specific location using the delivery methods described as follows. As a non-limiting example, a subject may be administered an empty polymeric particle prior to, simultaneously with or after delivering the shingles vaccine composition of the present disclosure to the subject. The empty polymeric particle undergoes a change in volume once in contact with the subject and becomes lodged, embedded, immobilized or entrapped at a specific location in the subject.

In another embodiment, the shingles vaccine composition of the present disclosure may be formulated in an active substance release system. For instance, the active substance release system may comprise at least one nanoparticle bonded to an oligonucleotide inhibitor strand which is hybridized with a catalytically active nucleic acid and a compound bonded to at least one substrate molecule bonded to a therapeutically active substance (e.g., polynucleotides described herein), where the therapeutically active substance is released by the cleavage of the substrate molecule by the catalytically active nucleic acid.

In another embodiment, the shingles vaccine composition of the present disclosure may be formulated in a nanoparticle comprising an inner core comprising a non-cellular material and an outer surface comprising a cellular membrane. The cellular membrane may be derived from a cell or a membrane derived from a virus.

In another embodiment, the shingles vaccine composition of the present disclosure may be formulated in porous nanoparticle-supported lipid bilayers (protocells).

In another embodiment, the shingles vaccine composition of the present disclosure may be formulated in polymeric nanoparticles which have a high glass transition temperature.

In another embodiment, the shingles vaccine composition of the present disclosure may be formulated in nanoparticles used in imaging. As a non-limiting example, the liposome may comprise gadolinium(III)2-{4,7-bis-carboxymethyl-10-[(N,N-distearylamidomethyl-N′-amido-methyl]-1,4,7,10-tetra-azacyclododec-1-yl}-acetic acid and a neutral, fully saturated phospholipid component.

The nanoparticles of the present disclosure may further include nutrients such as, but not limited to, those which deficiencies can lead to health hazards from anemia to neural tube defects. As a non-limiting example, the nutrient may be iron in the form of ferrous, ferric salts or elemental iron, iodine, folic acid, vitamins or micronutrients.

In another embodiment, the shingles vaccine composition of the present disclosure may be formulated in a swellable nanoparticle.

In another embodiment, the shingles vaccine composition of the present disclosure may be formulated in polyanhydride nanoparticles.

The nanoparticles and microparticles of the present disclosure may be geometrically engineered to modulate macrophage and/or the immune response. In some embodiments, the geometrically engineered particles may have varied shapes, sizes and/or surface charges in order to incorporated the polynucleotides of the present disclosure for targeted delivery such as, but not limited to, pulmonary delivery. Other physical features the geometrically engineering particles may have include, but are not limited to, fenestrations, angled arms, asymmetry and surface roughness, charge which can alter the interactions with cells and tissues.

In another embodiment, the nanoparticles of the present disclosure may be water soluble nanoparticles. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In some embodiments, the nanoparticles of the present disclosure are stealth nanoparticles or target-specific stealth nanoparticles. In some embodiments, the stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In one embodiment, the nanoparticle of the present disclosure may be a nanoparticle-nucleic acid hybrid structure having a high density nucleic acid layer. The nanoparticle of the present disclosure may comprise a nucleic acid such as, but not limited to, polynucleotides described herein and/or known in the art.

In one embodiment, at least one of the nanoparticles of the present disclosure may be embedded in in the core a nanostructure or coated with a low density porous 3-D structure or coating which is capable of carrying or associating with at least one payload within or on the surface of the nanostructure.

In one embodiment, the pharmaceutically acceptable carrier is a lipid nanoparticle encapsulating the mRNAs of the present disclosure therein. In another embodiment, the lipid nanoparticle comprises a first lipid nanoparticle encapsulating the mRNA encoding VZV gE (soluble), and a second lipid nanoparticle encapsulating the mRNA encoding VZV gE (full-length).

2. The Method of Inducing Immune Response Against Shingles

The present disclosure also provides a method of inducing immune response against shingles comprising administering an effective amount of the shingles vaccine composition of the present disclosure a subject in need thereof. In one embodiment, the effective amount of the shingles vaccine composition (e.g. mRNA) is provided based, at least in part, on the target tissue, target cell type, means of administration, physical characteristics of the polynucleotide (e.g., size, and extent of modified nucleosides) and other components of the vaccine, and other determinants. In general, an effective amount of the shingles vaccine (e.g., mRNA) provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the RNA, e.g., mRNA, vaccine), increased protein translation from the polynucleotide, decreased nucleic acid degradation (as demonstrated, for example, by increased duration of protein translation from a modified polynucleotide), or altered antigen specific immune response of the host cell.

Administration of an effective amount (immunogenically effective amount) of the shingles vaccine compositions (e.g., shingles vaccine compositions (1) and (2)) is typically intramuscular or subcutaneous. Thus, the shingles vaccine composition is typically formulated for intramuscular or subcutaneous injection, and for the purposes of the invention formulated without adjuvants, preferably without any adjuvant. However other modes of administration, such as intravenous, cutaneous, intradermal or nasal can be envisaged as well. For intravenous, cutaneous or subcutaneous injection, the adenovirus vector will be in the form of a parenterally acceptable aqueous solution which is pyrogen-free and has suitable pH, isotonicity and stability. Likewise, the isolated envelope polypeptide will be in the form of a parenterally acceptable solution having a suitable pH, isotonicity, and stability. Those of ordinary skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

In a particular embodiment, an effective amount (immunogenically effective amount) of the shingles vaccine composition (e.g., shingles vaccine compositions (1) and (2)) is administered via intramuscular administration. Intramuscular administration can be achieved by using a needle to inject a suspension of the adenovirus vectors and/or envelope polypeptides. An alternative is the use of a needleless injection device to administer the composition (using, e.g., Biojector™) or a freeze-dried powder containing the vaccine.

In one embodiment, the priming immunization and/or the boosting administration, preferably both the priming and boosting administration, further comprise administering one or more adenovirus vectors that encode one or more further shingles antigens.

The timing for administering priming and boosting immunizations is not particularly limited. For example, a vaccine composition can be administered for priming immunization, and re-administered prior to administration of a vaccine composition for boosting immunization. Further administrations of a vaccine composition for further boosting immunizations are also contemplated. In certain embodiments, a booster vaccine is first administered about 1-12 weeks, e.g., 1, 2, 3, 4, 5, 6, 7, 8, 9, 10, 11, or 12 weeks after a primer vaccine is initially administered. In other embodiments, a booster vaccine is first administered about 12-52 weeks, e.g., about 12, 14, 16, 18, 20, 22, 24, 26, 28, 30, 32, 34, 36, 38, 40, 42, 44, 46, 48, 50, or 52 weeks after a primer vaccine is initially administered. One of ordinary skill in the art will be able to vary the exact timing of the priming and boosting vaccines, frequency of administration thereof, dosage thereof, etc., based upon the teachings herein and general knowledge in the art.

In one embodiment, the shingles vaccine composition may comprise the first and second mRNAs described herein, formulated in a lipid nanoparticle comprising MC3, Cholesterol, DSPC and PEG2000-DMG, the buffer trisodium citrate, sucrose and water for injection. As a non-limiting example, the composition may comprise 2.0 mg/mL of drug substance (e.g., shingles vaccine compositions (1) and (2)), 21.8 mg/mL of MC3, 10.1 mg/mL of cholesterol, 5.4 mg/mL of DSPC, 2.7 mg/mL of PEG2000-DMG, 5.16 mg/mL of trisodium citrate, 71 mg/mL of sucrose and 1.0 mL of water for injection.

In one embodiment, a method of inducing immune response against shingles comprises administering an effective amount of the shingles vaccine composition (1) of the present disclosure to a subject in need thereof. In the shingles vaccine composition (1), the mRNA having the structure of 5′UTR-ORF encoding VZV gE (soluble)-3′UTR-poly (A) tail may have a nucleotide sequence of SEQ ID NO: 5 (or a nucleotide sequence having at least 80%, 85%, 90%, 95%, 96%, 97%, 98%, 99% or 99% identity to SEQ ID NO: 5).

In another embodiment, a method of inducing immune response against shingles comprises administering an effective amount of the shingles vaccine composition (2) of the present disclosure to a subject in need thereof. In the shingles vaccine composition (2), the mRNA having the structure of 5′UTR-ORF encoding VZV gE (full length)-3′UTR-poly (A) tail may have a nucleotide sequence of SEQ ID NO: 6 (or a nucleotide sequence having at least 80%, 85%, 90%, 95%, 96%, 97%, 98%, 99% or 99% identity to SEQ ID NO: 6).

3. Sequence Information

The specific sequence information of SEQ ID NOS: 1 to 8 cited in the present disclosure is as follows.

SEQ ID NO: 1
MGTVNKPVVGVLMGFGIITGTLRITNPVRASVLRYDDFHIDEDKLDTNSVYEPYYHSDH
AESSWVNRGESSRKAYDHNSPYIWPRNDYDGFLENAHEHHGVYNQGRGIDSGERLMQ
PTQMSAQEDLGDDTGIHVIPTLNGDDRHKIVNVDQRQYGDVFKGDLNPKPQGQRLIEVS
VEENHPFTLRAPIQRIYGVRYTETWSFLPSLTCTGDAAPAIQHICLKHTTCFQDVVVDVD
CAENTKEDQLAEISYRFQGKKEADQPWIVVNTSTLFDELELDPPEIEPGVLKVLRTEKQY
LGVYIWNMRGSDGTSTYATFLVTWKGDEKTRNPTPAVTPQPRGAEFHMWNYHSHVFS
VGDTFSLAMHLQYKIHEAPFDLLLEWLYVPIDPTCQPMRLYSTCLYHPNAPQCLSHMNS
GCTFTSPHLAQRVASTVYQNCEHADNYTAYCLGISHMEPSFGLILHDGGTTLKFVDTPE
SLSGLYVFVVYFNGHVEAVAYTVVSTVDHFVNAIEERGFPPTAGQPPATTKPKEITPVNP
GTSPLLRYAAWTGGLAA
2) Protein sequence of the ORF encoded in full-length VZV gE
SEQ ID NO: 2
MGTVNKPVVGVLMGFGIITGTLRITNPVRASVLRYDDFHIDEDKLDTNSVYEPYYHSDH
AESSWVNRGESSRKAYDHNSPYIWPRNDYDGFLENAHEHHGVYNQGRGIDSGERLMQ
PTQMSAQEDLGDDTGIHVIPTLNGDDRHKIVNVDQRQYGDVFKGDLNPKPQGQRLIEVS
VEENHPFTLRAPIQRIYGVRYTETWSFLPSLTCTGDAAPAIQHICLKHTTCFQDVVVDVD
CAENTKEDQLAEISYRFQGKKEADQPWIVVNTSTLFDELELDPPEIEPGVLKVLRTEKQY
LGVYIWNMRGSDGTSTYATFLVTWKGDEKTRNPTPAVTPQPRGAEFHMWNYHSHVFS
VGDTFSLAMHLQYKIHEAPFDLLLEWLYVPIDPTCQPMRLYSTCLYHPNAPQCLSHMNS
GCTFTSPHLAQRVASTVYQNCEHADNYTAYCLGISHMEPSFGLILHDGGTTLKFVDTPE
SLSGLYVFVVYFNGHVEAVAYTVVSTVDHFVNAIEERGFPPTAGQPPATTKPKEITPVNP
GTSPLLRYAAWTGGLAAVVLLCLVIFLICTAKRMRVKAYRVDKSPYNQSMYYAGLPVD
DFEDSESTDTEEEFGNAIGGSHGGSSYTVYIDKTR
3) Soluble VZV gE mRNA sequence (ORF)
SEQ ID NO: 3
AUGGGCACCGUGAAUAAACCUGUGGUGGGGGUAUUGAUGGGGUUCGGAAUUAUC
ACGGGAACGUUGCGUAUAACGAAUCCGGUCAGAGCAUCCGUCUUGCGAUACGAU
GAUUUUCACAUCGAUGAAGACAAACUGGAUACAAACUCCGUAUAUGAGCCUUAC
UACCAUUCAGAUCAUGCGGAGUCUUCAUGGGUAAAUCGGGGAGAGUCUUCGCGA
AAAGCGUACGAUCAUAACUCACCUUAUAUAUGGCCACGUAAUGAUUAUGAUGGA
UUUUUAGAGAACGCACACGAACACCAUGGGGUGUAUAAUCAGGGCCGUGGUAUC
GAUAGCGGGGAACGGUUAAUGCAACCCACACAAAUGUCUGCACAGGAGGAUCUU
GGGGACGAUACGGGCAUCCACGUUAUCCCUACGUUAAACGGCGAUGACAGACAUA
AAAUUGUAAAUGUGGACCAACGUCAAUACGGUGACGUGUUUAAAGGAGAUCUUA
AUCCAAAACCCCAAGGCCAAAGACUCAUUGAGGUGUCAGUGGAAGAAAAUCACCC
GUUUACUUUACGCGCACCGAUUCAGCGGAUUUAUGGAGUCCGGUACACCGAGACU
UGGAGCUUUUUGCCGUCAUUAACCUGUACGGGAGACGCAGCGCCCGCCAUCCAGC
AUAUAUGUUUAAAACAUACAACAUGCUUUCAAGACGUGGUGGUGGAUGUGGAUU
GCGCGGAAAAUACUAAAGAGGAUCAGUUGGCCGAAAUCAGUUACCGUUUUCAAG
GUAAGAAGGAAGCGGACCAACCGUGGAUUGUUGUAAACACGAGCACACUGUUUG
AUGAACUCGAAUUAGACCCCCCCGAGAUUGAACCGGGUGUCUUGAAAGUACUUCG
GACAGAAAAACAAUACUUGGGUGUGUACAUUUGGAACAUGCGCGGCUCCGAUGG
UACGUCUACCUACGCCACGUUUUUGGUCACCUGGAAAGGGGAUGAAAAAACAAG
AAACCCUACGCCCGCAGUAACUCCUCAACCAAGAGGGGCUGAGUUUCAUAUGUGG
AAUUACCACUCGCAUGUAUUUUCAGUUGGUGAUACGUUUAGCUUGGCAAUGCAU
CUUCAGUAUAAGAUACAUGAAGCGCCAUUUGAUUUGCUGUUAGAGUGGUUGUAU
GUCCCCAUCGAUCCUACAUGUCAACCAAUGCGGUUAUAUUCUACGUGUUUGUAUC
AUCCCAACGCACCCCAAUGCCUCUCUCAUAUGAAUUCCGGUUGUACAUUUACCUC
GCCACAUUUAGCCCAGCGUGUUGCAAGCACAGUGUAUCAAAAUUGUGAACAUGC
AGAUAACUACACCGCAUAUUGUCUGGGAAUAUCUCAUAUGGAGCCUAGCUUUGG
UCUAAUCUUACACGACGGGGGCACCACGUUAAAGUUUGUAGAUACACCCGAGAG
UUUGUCGGGAUUAUACGUUUUUGUGGUGUAUUUUAACGGGCAUGUUGAAGCCGU
AGCAUACACUGUUGUAUCCACAGUAGAUCAUUUUGUAAACGCAAUUGAGGAGCG
UGGAUUUCCGCCAACGGCCGGUCAGCCACCGGCGACUACUAAACCCAAGGAAAUU
ACCCCCGUAAACCCCGGAACGUCACCACUUCUACGAUAUGCCGCAUGGACCGGAG
GGCUUGCAGCA
4) Full-length VZV gE mRNA sequence (ORF)
SEQ ID NO: 4
AUGGGCACCGUGAAUAAACCUGUGGUGGGGGUAUUGAUGGGGUUCGGAAUUAUC
ACGGGAACGUUGCGUAUAACGAAUCCGGUCAGAGCAUCCGUCUUGCGAUACGAU
GAUUUUCACAUCGAUGAAGACAAACUGGAUACAAACUCCGUAUAUGAGCCUUAC
UACCAUUCAGAUCAUGCGGAGUCUUCAUGGGUAAAUCGGGGAGAGUCUUCGCGA
AAAGCGUACGAUCAUAACUCACCUUAUAUAUGGCCACGUAAUGAUUAUGAUGGA
UUUUUAGAGAACGCACACGAACACCAUGGGGUGUAUAAUCAGGGCCGUGGUAUC
GAUAGCGGGGAACGGUUAAUGCAACCCACACAAAUGUCUGCACAGGAGGAUCUU
GGGGACGAUACGGGCAUCCACGUUAUCCCUACGUUAAACGGCGAUGACAGACAUA
AAAUUGUAAAUGUGGACCAACGUCAAUACGGUGACGUGUUUAAAGGAGAUCUUA
AUCCAAAACCCCAAGGCCAAAGACUCAUUGAGGUGUCAGUGGAAGAAAAUCACCC
GUUUACUUUACGCGCACCGAUUCAGCGGAUUUAUGGAGUCCGGUACACCGAGACU
UGGAGCUUUUUGCCGUCAUUAACCUGUACGGGAGACGCAGCGCCCGCCAUCCAGC
AUAUAUGUUUAAAACAUACAACAUGCUUUCAAGACGUGGUGGUGGAUGUGGAUU
GCGCGGAAAAUACUAAAGAGGAUCAGUUGGCCGAAAUCAGUUACCGUUUUCAAG
GUAAGAAGGAAGCGGACCAACCGUGGAUUGUUGUAAACACGAGCACACUGUUUG
AUGAACUCGAAUUAGACCCCCCCGAGAUUGAACCGGGUGUCUUGAAAGUACUUCG
GACAGAAAAACAAUACUUGGGUGUGUACAUUUGGAACAUGCGCGGCUCCGAUGG
UACGUCUACCUACGCCACGUUUUUGGUCACCUGGAAAGGGGAUGAAAAAACAAG
AAACCCUACGCCCGCAGUAACUCCUCAACCAAGAGGGGCUGAGUUUCAUAUGUGG
AAUUACCACUCGCAUGUAUUUUCAGUUGGUGAUACGUUUAGCUUGGCAAUGCAU
CUUCAGUAUAAGAUACAUGAAGCGCCAUUUGAUUUGCUGUUAGAGUGGUUGUAU
GUCCCCAUCGAUCCUACAUGUCAACCAAUGCGGUUAUAUUCUACGUGUUUGUAUC
AUCCCAACGCACCCCAAUGCCUCUCUCAUAUGAAUUCCGGUUGUACAUUUACCUC
GCCACAUUUAGCCCAGCGUGUUGCAAGCACAGUGUAUCAAAAUUGUGAACAUGC
AGAUAACUACACCGCAUAUUGUCUGGGAAUAUCUCAUAUGGAGCCUAGCUUUGG
UCUAAUCUUACACGACGGGGGCACCACGUUAAAGUUUGUAGAUACACCCGAGAG
UUUGUCGGGAUUAUACGUUUUUGUGGUGUAUUUUAACGGGCAUGUUGAAGCCGU
AGCAUACACUGUUGUAUCCACAGUAGAUCAUUUUGUAAACGCAAUUGAGGAGCG
UGGAUUUCCGCCAACGGCCGGUCAGCCACCGGCGACUACUAAACCCAAGGAAAUU
ACCCCCGUAAACCCCGGAACGUCACCACUUCUACGAUAUGCCGCAUGGACCGGAG
GGCUUGCAGCAGUAGUACUUUUAUGUCUCGUAAUAUUUUUAAUCUGUACGGCUA
AACGAAUGAGGGUUAAAGCCUAUAGGGUAGACAAGUCCCCGUAUAACCAAAGCA
UGUAUUACGCUGGCCUUCCAGUGGACGAUUUCGAGGACUCGGAAUCUACGGAUA
CGGAAGAAGAGUUUGGUAACGCGAUUGGAGGGAGUCACGGGGGUUCGAGUUACA
CGGUGUAUAUAGAUAAGACCCGG
5) Soluble VZV gE mRNA sequence (5′UTR-ORF-3′UTR-poly
(A) tail)
SEQ ID NO: 5
AGGCCGGCACUCUUCUGGUCCCCACAGACUCAGAGAGAACCCGCCGCCACCAUGG
GCACCGUGAAUAAACCUGUGGUGGGGGUAUUGAUGGGGUUCGGAAUUAUCACGG
GAACGUUGCGUAUAACGAAUCCGGUCAGAGCAUCCGUCUUGCGAUACGAUGAUU
UUCACAUCGAUGAAGACAAACUGGAUACAAACUCCGUAUAUGAGCCUUACUACCA
UUCAGAUCAUGCGGAGUCUUCAUGGGUAAAUCGGGGAGAGUCUUCGCGAAAAGC
GUACGAUCAUAACUCACCUUAUAUAUGGCCACGUAAUGAUUAUGAUGGAUUUUU
AGAGAACGCACACGAACACCAUGGGGUGUAUAAUCAGGGCCGUGGUAUCGAUAG
CGGGGAACGGUUAAUGCAACCCACACAAAUGUCUGCACAGGAGGAUCUUGGGGA
CGAUACGGGCAUCCACGUUAUCCCUACGUUAAACGGCGAUGACAGACAUAAAAUU
GUAAAUGUGGACCAACGUCAAUACGGUGACGUGUUUAAAGGAGAUCUUAAUCCA
AAACCCCAAGGCCAAAGACUCAUUGAGGUGUCAGUGGAAGAAAAUCACCCGUUU
ACUUUACGCGCACCGAUUCAGCGGAUUUAUGGAGUCCGGUACACCGAGACUUGGA
GCUUUUUGCCGUCAUUAACCUGUACGGGAGACGCAGCGCCCGCCAUCCAGCAUAU
AUGUUUAAAACAUACAACAUGCUUUCAAGACGUGGUGGUGGAUGUGGAUUGCGC
GGAAAAUACUAAAGAGGAUCAGUUGGCCGAAAUCAGUUACCGUUUUCAAGGUAA
GAAGGAAGCGGACCAACCGUGGAUUGUUGUAAACACGAGCACACUGUUUGAUGA
ACUCGAAUUAGACCCCCCCGAGAUUGAACCGGGUGUCUUGAAAGUACUUCGGACA
GAAAAACAAUACUUGGGUGUGUACAUUUGGAACAUGCGCGGCUCCGAUGGUACG
UCUACCUACGCCACGUUUUUGGUCACCUGGAAAGGGGAUGAAAAAACAAGAAAC
CCUACGCCCGCAGUAACUCCUCAACCAAGAGGGGCUGAGUUUCAUAUGUGGAAUU
ACCACUCGCAUGUAUUUUCAGUUGGUGAUACGUUUAGCUUGGCAAUGCAUCUUC
AGUAUAAGAUACAUGAAGCGCCAUUUGAUUUGCUGUUAGAGUGGUUGUAUGUCC
CCAUCGAUCCUACAUGUCAACCAAUGCGGUUAUAUUCUACGUGUUUGUAUCAUCC
CAACGCACCCCAAUGCCUCUCUCAUAUGAAUUCCGGUUGUACAUUUACCUCGCCA
CAUUUAGCCCAGCGUGUUGCAAGCACAGUGUAUCAAAAUUGUGAACAUGCAGAU
AACUACACCGCAUAUUGUCUGGGAAUAUCUCAUAUGGAGCCUAGCUUUGGUCUA
AUCUUACACGACGGGGGCACCACGUUAAAGUUUGUAGAUACACCCGAGAGUUUG
UCGGGAUUAUACGUUUUUGUGGUGUAUUUUAACGGGCAUGUUGAAGCCGUAGCA
UACACUGUUGUAUCCACAGUAGAUCAUUUUGUAAACGCAAUUGAGGAGCGUGGA
UUUCCGCCAACGGCCGGUCAGCCACCGGCGACUACUAAACCCAAGGAAAUUACCC
CCGUAAACCCCGGAACGUCACCACUUCUACGAUAUGCCGCAUGGACCGGAGGGCU
UGCAGCAUGAUAAAGCUGGAGCCUCGGUGGCCUUGCUUCUUGCCCCUUGGGCCUC
CCCCCAGCCCCUCCUCCCCUUCCUGCACCCGUACCCCCGUGGUCUUUGAAUAAAGU
CUGAGUGGGCGGCAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAA
6) Full-length VZV gE mRNA sequence (5′UTR-ORF-3′UTR-
poly (A) tail)
SEQ ID NO: 6
AGGCCGGCACUCUUCUGGUCCCCACAGACUCAGAGAGAACCCGCCGCCACCAUGG
GCACCGUGAAUAAACCUGUGGUGGGGGUAUUGAUGGGGUUCGGAAUUAUCACGG
GAACGUUGCGUAUAACGAAUCCGGUCAGAGCAUCCGUCUUGCGAUACGAUGAUU
UUCACAUCGAUGAAGACAAACUGGAUACAAACUCCGUAUAUGAGCCUUACUACCA
UUCAGAUCAUGCGGAGUCUUCAUGGGUAAAUCGGGGAGAGUCUUCGCGAAAAGC
GUACGAUCAUAACUCACCUUAUAUAUGGCCACGUAAUGAUUAUGAUGGAUUUUU
AGAGAACGCACACGAACACCAUGGGGUGUAUAAUCAGGGCCGUGGUAUCGAUAG
CGGGGAACGGUUAAUGCAACCCACACAAAUGUCUGCACAGGAGGAUCUUGGGGA
CGAUACGGGCAUCCACGUUAUCCCUACGUUAAACGGCGAUGACAGACAUAAAAUU
GUAAAUGUGGACCAACGUCAAUACGGUGACGUGUUUAAAGGAGAUCUUAAUCCA
AAACCCCAAGGCCAAAGACUCAUUGAGGUGUCAGUGGAAGAAAAUCACCCGUUU
ACUUUACGCGCACCGAUUCAGCGGAUUUAUGGAGUCCGGUACACCGAGACUUGGA
GCUUUUUGCCGUCAUUAACCUGUACGGGAGACGCAGCGCCCGCCAUCCAGCAUAU
AUGUUUAAAACAUACAACAUGCUUUCAAGACGUGGUGGUGGAUGUGGAUUGCGC
GGAAAAUACUAAAGAGGAUCAGUUGGCCGAAAUCAGUUACCGUUUUCAAGGUAA
GAAGGAAGCGGACCAACCGUGGAUUGUUGUAAACACGAGCACACUGUUUGAUGA
ACUCGAAUUAGACCCCCCCGAGAUUGAACCGGGUGUCUUGAAAGUACUUCGGACA
GAAAAACAAUACUUGGGUGUGUACAUUUGGAACAUGCGCGGCUCCGAUGGUACG
UCUACCUACGCCACGUUUUUGGUCACCUGGAAAGGGGAUGAAAAAACAAGAAAC
CCUACGCCCGCAGUAACUCCUCAACCAAGAGGGGCUGAGUUUCAUAUGUGGAAUU
ACCACUCGCAUGUAUUUUCAGUUGGUGAUACGUUUAGCUUGGCAAUGCAUCUUC
AGUAUAAGAUACAUGAAGCGCCAUUUGAUUUGCUGUUAGAGUGGUUGUAUGUCC
CCAUCGAUCCUACAUGUCAACCAAUGCGGUUAUAUUCUACGUGUUUGUAUCAUCC
CAACGCACCCCAAUGCCUCUCUCAUAUGAAUUCCGGUUGUACAUUUACCUCGCCA
CAUUUAGCCCAGCGUGUUGCAAGCACAGUGUAUCAAAAUUGUGAACAUGCAGAU
AACUACACCGCAUAUUGUCUGGGAAUAUCUCAUAUGGAGCCUAGCUUUGGUCUA
AUCUUACACGACGGGGGCACCACGUUAAAGUUUGUAGAUACACCCGAGAGUUUG
UCGGGAUUAUACGUUUUUGUGGUGUAUUUUAACGGGCAUGUUGAAGCCGUAGCA
UACACUGUUGUAUCCACAGUAGAUCAUUUUGUAAACGCAAUUGAGGAGCGUGGA
UUUCCGCCAACGGCCGGUCAGCCACCGGCGACUACUAAACCCAAGGAAAUUACCC
CCGUAAACCCCGGAACGUCACCACUUCUACGAUAUGCCGCAUGGACCGGAGGGCU
UGCAGCAGUAGUACUUUUAUGUCUCGUAAUAUUUUUAAUCUGUACGGCUAAACG
AAUGAGGGUUAAAGCCUAUAGGGUAGACAAGUCCCCGUAUAACCAAAGCAUGUA
UUACGCUGGCCUUCCAGUGGACGAUUUCGAGGACUCGGAAUCUACGGAUACGGA
AGAAGAGUUUGGUAACGCGAUUGGAGGGAGUCACGGGGGUUCGAGUUACACGGU
GUAUAUAGAUAAGACCCGGUGAUAAAGCUGGAGCCUCGGUGGCCUUGCUUCUUG
CCCCUUGGGCCUCCCCCCAGCCCCUCCUCCCCUUCCUGCACCCGUACCCCCGUGGU
CUUUGAAUAAAGUCUGAGUGGGGGCAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAA
7) pUC57-Kan plasmid encoding soluble VZV gE mRNA
(soluble VZV gE mRNA sequence is underlined)
SEQ ID NO: 7
TCGCGCGTTTCGGTGATGACGGTGAAAACCTCTGACACATGCAGCTCCCGGAGACG
GTCACAGCTTGTCTGTAAGCGGATGCCGGGAGCAGACAAGCCCGTCAGGGCGCGTC
AGCGGGTGTTGGCGGGTGTCGGGGCTGGCTTAACTATGCGGCATCAGAGCAGATTG
TACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCA
GAGAATTCGAGCTCGGTACCTCGCGAATACATCTAGATTAATACGACTCACTATAAG
GCCGGCACTCTTCTGGTCCCCACAGACTCAGAGAGAACCCGCCGCCACCATGGGCA
CCGTGAATAAACCTGTGGTGGGGGTATTGATGGGGTTCGGAATTATCACGGGAACG
TTGCGTATAACGAATCCGGTCAGAGCATCCGTCTTGCGATACGATGATTTTCACATC
GATGAAGACAAACTGGATACAAACTCCGTATATGAGCCTTACTACCATTCAGATCAT
GCGGAGTCTTCATGGGTAAATCGGGGAGAGTCTTCGCGAAAAGCGTACGATCATAA
CTCACCTTATATATGGCCACGTAATGATTATGATGGATTTTTAGAGAACGCACACGA
ACACCATGGGGTGTATAATCAGGGCCGTGGTATCGATAGCGGGGAACGGTTAATGC
AACCCACACAAATGTCTGCACAGGAGGATCTTGGGGACGATACGGGCATCCACGTT
ATCCCTACGTTAAACGGCGATGACAGACATAAAATTGTAAATGTGGACCAACGTCA
ATACGGTGACGTGTTTAAAGGAGATCTTAATCCAAAACCCCAAGGCCAAAGACTCA
TTGAGGTGTCAGTGGAAGAAAATCACCCGTTTACTTTACGCGCACCGATTCAGCGGA
TTTATGGAGTCCGGTACACCGAGACTTGGAGCTTTTTGCCGTCATTAACCTGTACGG
GAGACGCAGCGCCCGCCATCCAGCATATATGTTTAAAACATACAACATGCTTTCAAG
ACGTGGTGGTGGATGTGGATTGCGCGGAAAATACTAAAGAGGATCAGTTGGCCGAA
ATCAGTTACCGTTTTCAAGGTAAGAAGGAAGCGGACCAACCGTGGATTGTTGTAAA
CACGAGCACACTGTTTGATGAACTCGAATTAGACCCCCCCGAGATTGAACCGGGTGT
CTTGAAAGTACTTCGGACAGAAAAACAATACTTGGGTGTGTACATTTGGAACATGCG
CGGCTCCGATGGTACGTCTACCTACGCCACGTTTTTGGTCACCTGGAAAGGGGATGA
AAAAACAAGAAACCCTACGCCCGCAGTAACTCCTCAACCAAGAGGGGCTGAGTTTC
ATATGTGGAATTACCACTCGCATGTATTTTCAGTTGGTGATACGTTTAGCTTGGCAAT
GCATCTTCAGTATAAGATACATGAAGCGCCATTTGATTTGCTGTTAGAGTGGTTGTA
TGTCCCCATCGATCCTACATGTCAACCAATGCGGTTATATTCTACGTGTTTGTATCAT
CCCAACGCACCCCAATGCCTCTCTCATATGAATTCCGGTTGTACATTTACCTCGCCAC
ATTTAGCCCAGCGTGTTGCAAGCACAGTGTATCAAAATTGTGAACATGCAGATAACT
ACACCGCATATTGTCTGGGAATATCTCATATGGAGCCTAGCTTTGGTCTAATCTTAC
ACGACGGGGGCACCACGTTAAAGTTTGTAGATACACCCGAGAGTTTGTCGGGATTAT
ACGTTTTTGTGGTGTATTTTAACGGGCATGTTGAAGCCGTAGCATACACTGTTGTATC
CACAGTAGATCATTTTGTAAACGCAATTGAGGAGCGTGGATTTCCGCCAACGGCCGG
TCAGCCACCGGCGACTACTAAACCCAAGGAAATTACCCCCGTAAACCCCGGAACGT
CACCACTTCTACGATATGCCGCATGGACCGGAGGGCTTGCAGCATGATAAAGCTGG
AGCCTCGGTGGCCTTGCTTCTTGCCCCTTGGGCCTCCCCCCAGCCCCTCCTCCCCTTC
CTGCACCCGTACCCCCGTGGTCTTTGAATAAAGTCTGAGTGGGCGGCAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAATGAAGAGCATCGGATCCCGGGCCCGTCGACTGCAGAGGCCTGCATGCAAGCTT
GGTGTAATCATGGTCATAGCTGTTTCCTGTGTGAAATTGTTATCCGCTCACAATTCCA
CACAACATACGAGCCGGAAGCATAAAGTGTAAAGCCTGGGGTGCCTAATGAGTGAG
CTAACTCACATTAATTGCGTTGCGCTCACTGCCCGCTTTCCAGTCGGGAAACCTGTC
GTGCCAGCTGCATTAATGAATCGGCCAACGCGCGGGGAGAGGCGGTTTGCGTATTG
GGCGCTCTTCCGCTTCCTCGCTCACTGACTCGCTGCGCTCGGTCGTTCGGCTGCGGCG
AGCGGTATCAGCTCACTCAAAGGCGGTAATACGGTTATCCACAGAATCAGGGGATA
ACGCAGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTA
AGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAG
AAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTT
CTACGGGGTCTGACGCTCAGTGGAACGAAAACTCACGTTAAGGGATTTTGGTCATGA
GATTATCAAAAAGGATCTTCACCTAGATCCTTTTAAATTAAAAATGAAGTTTTAAAT
CAAGCCCAATCTGAATAATGTTACAACCAATTAACCAATTCTGATTAGAAAAACTCA
TCGAGCATCAAATGAAACTGCAATTTATTCATATCAGGATTATCAATACCATATTTTT
GAAAAAGCCGTTTCTGTAATGAAGGAGAAAACTCACCGAGGCAGTTCCATAGGATG
GCAAGATCCTGGTATCGGTCTGCGATTCCGACTCGTCCAACATCAATACAACCTATT
AATTTCCCCTCGTCAAAAATAAGGTTATCAAGTGAGAAATCACCATGAGTGACGACT
GAATCCGGTGAGAATGGCAAAAGTTTATGCATTTCTTTCCAGACTTGTTCAACAGGC
CAGCCATTACGCTCGTCATCAAAATCACTCGCATCAACCAAACCGTTATTCATTCGT
GATTGCGCCTGAGCGAGACGAAATACGCGATCGCTGTTAAAAGGACAATTACAAAC
AGGAATCGAATGCAACCGGCGCAGGAACACTGCCAGCGCATCAACAATATTTTCAC
CTGAATCAGGATATTCTTCTAATACCTGGAATGCTGTTTTTCCGGGGATCGCAGTGG
TGAGTAACCATGCATCATCAGGAGTACGGATAAAATGCTTGATGGTCGGAAGAGGC
ATAAATTCCGTCAGCCAGTTTAGTCTGACCATCTCATCTGTAACATCATTGGCAACG
CTACCTTTGCCATGTTTCAGAAACAACTCTGGCGCATCGGGCTTCCCATACAAGCGA
TAGATTGTCGCACCTGATTGCCCGACATTATCGCGAGCCCATTTATACCCATATAAA
TCAGCATCCATGTTGGAATTTAATCGCGGCCTCGACGTTTCCCGTTGAATATGGCTC
ATAACACCCCTTGTATTACTGTTTATGTAAGCAGACAGTTTTATTGTTCATGATGATA
TATTTTTATCTTGTGCAATGTAACATCAGAGATTTTGAGACACGGGCCAGAGCTGCA
8) pUC57-Kan plasmid encoding full-length VZV gE mRNA
(full-length VZV gE mRNA sequence is underlined)
SEQ ID NO: 8
TCGCGCGTTTCGGTGATGACGGTGAAAACCTCTGACACATGCAGCTCCCGGAGACG
GTCACAGCTTGTCTGTAAGCGGATGCCGGGAGCAGACAAGCCCGTCAGGGCGCGTC
AGCGGGTGTTGGCGGGTGTCGGGGCTGGCTTAACTATGCGGCATCAGAGCAGATTG
TACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCA
GAGAATTCGAGCTCGGTACCTCGCGAATACATCTAGATTAATACGACTCACTATAAG
GCCGGCACTCTTCTGGTCCCCACAGACTCAGAGAGAACCCGCCGCCACCATGGGCA
CCGTGAATAAACCTGTGGTGGGGGTATTGATGGGGTTCGGAATTATCACGGGAACG
TTGCGTATAACGAATCCGGTCAGAGCATCCGTCTTGCGATACGATGATTTTCACATC
GATGAAGACAAACTGGATACAAACTCCGTATATGAGCCTTACTACCATTCAGATCAT
GCGGAGTCTTCATGGGTAAATCGGGGAGAGTCTTCGCGAAAAGCGTACGATCATAA
CTCACCTTATATATGGCCACGTAATGATTATGATGGATTTTTAGAGAACGCACACGA
ACACCATGGGGTGTATAATCAGGGCCGTGGTATCGATAGCGGGGAACGGTTAATGC
AACCCACACAAATGTCTGCACAGGAGGATCTTGGGGACGATACGGGCATCCACGTT
ATCCCTACGTTAAACGGCGATGACAGACATAAAATTGTAAATGTGGACCAACGTCA
ATACGGTGACGTGTTTAAAGGAGATCTTAATCCAAAACCCCAAGGCCAAAGACTCA
TTGAGGTGTCAGTGGAAGAAAATCACCCGTTTACTTTACGCGCACCGATTCAGCGGA
TTTATGGAGTCCGGTACACCGAGACTTGGAGCTTTTTGCCGTCATTAACCTGTACGG
GAGACGCAGCGCCCGCCATCCAGCATATATGTTTAAAACATACAACATGCTTTCAAG
ACGTGGTGGTGGATGTGGATTGCGCGGAAAATACTAAAGAGGATCAGTTGGCCGAA
ATCAGTTACCGTTTTCAAGGTAAGAAGGAAGCGGACCAACCGTGGATTGTTGTAAA
CACGAGCACACTGTTTGATGAACTCGAATTAGACCCCCCCGAGATTGAACCGGGTGT
CTTGAAAGTACTTCGGACAGAAAAACAATACTTGGGTGTGTACATTTGGAACATGCG
CGGCTCCGATGGTACGTCTACCTACGCCACGTTTTTGGTCACCTGGAAAGGGGATGA
AAAAACAAGAAACCCTACGCCCGCAGTAACTCCTCAACCAAGAGGGGCTGAGTTTC
ATATGTGGAATTACCACTCGCATGTATTTTCAGTTGGTGATACGTTTAGCTTGGCAAT
GCATCTTCAGTATAAGATACATGAAGCGCCATTTGATTTGCTGTTAGAGTGGTTGTA
TGTCCCCATCGATCCTACATGTCAACCAATGCGGTTATATTCTACGTGTTTGTATCAT
CCCAACGCACCCCAATGCCTCTCTCATATGAATTCCGGTTGTACATTTACCTCGCCAC
ATTTAGCCCAGCGTGTTGCAAGCACAGTGTATCAAAATTGTGAACATGCAGATAACT
ACACCGCATATTGTCTGGGAATATCTCATATGGAGCCTAGCTTTGGTCTAATCTTAC
ACGACGGGGGCACCACGTTAAAGTTTGTAGATACACCCGAGAGTTTGTCGGGATTAT
ACGTTTTTGTGGTGTATTTTAACGGGCATGTTGAAGCCGTAGCATACACTGTTGTATC
CACAGTAGATCATTTTGTAAACGCAATTGAGGAGCGTGGATTTCCGCCAACGGCCGG
TCAGCCACCGGCGACTACTAAACCCAAGGAAATTACCCCCGTAAACCCCGGAACGT
CACCACTTCTACGATATGCCGCATGGACCGGAGGGCTTGCAGCAGTAGTACTTTTAT
GTCTCGTAATATTTTTAATCTGTACGGCTAAACGAATGAGGGTTAAAGCCTATAGGG
TAGACAAGTCCCCGTATAACCAAAGCATGTATTACGCTGGCCTTCCAGTGGACGATT
TCGAGGACTCGGAATCTACGGATACGGAAGAAGAGTTTGGTAACGCGATTGGAGGG
AGTCACGGGGGTTCGAGTTACACGGTGTATATAGATAAGACCCGGTGATAAAGCTG
GAGCCTCGGTGGCCTTGCTTCTTGCCCCTTGGGCCTCCCCCCAGCCCCTCCTCCCCTT
CCTGCACCCGTACCCCCGTGGTCTTTGAATAAAGTCTGAGTGGGCGGCAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAATGAAGAGCATCGGATCCCGGGCCCGTCGACTGCAGAGGCCTGCAT
GCAAGCTTGGTGTAATCATGGTCATAGCTGTTTCCTGTGTGAAATTGTTATCCGCTCA
CAATTCCACACAACATACGAGCCGGAAGCATAAAGTGTAAAGCCTGGGGTGCCTAA
TGAGTGAGCTAACTCACATTAATTGCGTTGCGCTCACTGCCCGCTTTCCAGTCGGGA
AACCTGTCGTGCCAGCTGCATTAATGAATCGGCCAACGCGCGGGGAGAGGCGGTTT
GCGTATTGGGCGCTCTTCCGCTTCCTCGCTCACTGACTCGCTGCGCTCGGTCGTTCGG
CTGCGGCGAGCGGTATCAGCTCACTCAAAGGCGGTAATACGGTTATCCACAGAATC
AGGGGATAACGCAGGAAAGAACATGTGAGCAAAAGGCCAGCAAAAGGCCAGGAAC
CGTAAAAAGGCCGCGTTGCTGGCGTTTTTCCATAGGCTCCGCCCCCCTGACGAGCAT
CACAAAAATCGACGCTCAAGTCAGAGGTGGCGAAACCCGACAGGACTATAAAGATA
CCAGGCGTTTCCCCCTGGAAGCTCCCTCGTGCGCTCTCCTGTTCCGACCCTGCCGCTT
ACCGGATACCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGTGTGCACG
AACCCCCCGTTCAGCCCGACCGCTGCGCCTTATCCGGTAACTATCGTCTTGAGTCCA
ACCCGGTAAGACACGACTTATCGCCACTGGCAGCAGCCACTGGTAACAGGATTAGC
AGAGCGAGGTATGTAGGCGGTGCTACAGAGTTCTTGAAGTGGTGGCCTAACTACGG
CTACACTAGAAGAACAGTATTTGGTATCTGCGCTCTGCTGAAGCCAGTTACCTTCGG
AAAAAGAGTTGGTAGCTCTTGATCCGGCAAACAAACCACCGCTGGTAGCGGTGGTT
TTTTTGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGGATTT
TGGTCATGAGATTATCAAAAAGGATCTTCACCTAGATCCTTTTAAATTAAAAATGAA
GTTTTAAATCAAGCCCAATCTGAATAATGTTACAACCAATTAACCAATTCTGATTAG
AAAAACTCATCGAGCATCAAATGAAACTGCAATTTATTCATATCAGGATTATCAATA
CCATATTTTTGAAAAAGCCGTTTCTGTAATGAAGGAGAAAACTCACCGAGGCAGTTC
CATAGGATGGCAAGATCCTGGTATCGGTCTGCGATTCCGACTCGTCCAACATCAATA
CAACCTATTAATTTCCCCTCGTCAAAAATAAGGTTATCAAGTGAGAAATCACCATGA
GTGACGACTGAATCCGGTGAGAATGGCAAAAGTTTATGCATTTCTTTCCAGACTTGT
TCAACAGGCCAGCCATTACGCTCGTCATCAAAATCACTCGCATCAACCAAACCGTTA
TTCATTCGTGATTGCGCCTGAGCGAGACGAAATACGCGATCGCTGTTAAAAGGACA
ATTACAAACAGGAATCGAATGCAACCGGCGCAGGAACACTGCCAGCGCATCAACAA
TATTTTCACCTGAATCAGGATATTCTTCTAATACCTGGAATGCTGTTTTTCCGGGGAT
CGCAGTGGTGAGTAACCATGCATCATCAGGAGTACGGATAAAATGCTTGATGGTCG
GAAGAGGCATAAATTCCGTCAGCCAGTTTAGTCTGACCATCTCATCTGTAACATCAT
TGGCAACGCTACCTTTGCCATGTTTCAGAAACAACTCTGGCGCATCGGGCTTCCCAT
ACAAGCGATAGATTGTCGCACCTGATTGCCCGACATTATCGCGAGCCCATTTATACC
CATATAAATCAGCATCCATGTTGGAATTTAATCGCGGCCTCGACGTTTCCCGTTGAA
TATGGCTCATAACACCCCTTGTATTACTGTTTATGTAAGCAGACAGTTTTATTGTTCA
TGATGATATATTTTTATCTTGTGCAATGTAACATCAGAGATTTTGAGACACGGGCCA
GAGCTGCA

### EXAMPLES

### Example 1—DNA Templates Used for In Vitro Transcription and Protein Expression

DNA template sequence for mRNA in vitro transcription (IVT) consists of T7 promoter, 5′ untranslated region (UTR), open reading frame (ORF) of glycoprotein E (gE) modified from the VZV ORF68 from oka strain DNA (GenBank: AH010548.2), 3′UTR and 120 bases of poly adenine (polyA). 5′UTR and 3′UTR are from human hemoglobin subunit alpha 1 (HBA1) mRNA (GenBank: NM_000558.5). For soluble form of glycoprotein E (gE sol), the transmembrane domain and Carboxy terminal was removed from the full length gE sequence, resulting in a secreted form (Δ Trp541-Arg623). All the DNA fragments were synthesized and subcloned into pUC57-Kan vector by GenScript (Piscataway, NJ). The Sequence of pUC57-Kan plasmid encoding soluble VZV gE mRNA is shown in SEQ ID NO: 7. The sequence of pUC57-Kan plasmid encoding full-length VZV gE mRNA was shown in SEQ ID NO: 8.

### Example 2—In Vitro Transcription (IVT)

The plasmid vector was linearized by restriction enzyme, B spQI (New England Biolabs) for gE and its soluble form. N1-Methylpseudouridine (m1Ψ) was purchase from BOC Sciences (Shirley, NY). IVT condition is followed by manufacture's recommendation (TranscriptAid T7 High Yield Transcription Kit, ThermoFisher) as below:
ATP/CTP/GTP/m1ψTP: 5 mM each
SmartCap (SC101, ST Pharm): 4 mM
Linear template DNA: 1 ug of plasmid
T7 RNA polymerase enzyme mix: 2 ul

IVT was carried out in 20 ul reaction incubated at 37° C. for 2 hours. The template DNA is removed by 2 units of DNase I (Invitrogen) treated at 37° C. for 15 min followed by a column purification (Monarch RNA Cleanup Kit, New England Biolabs).

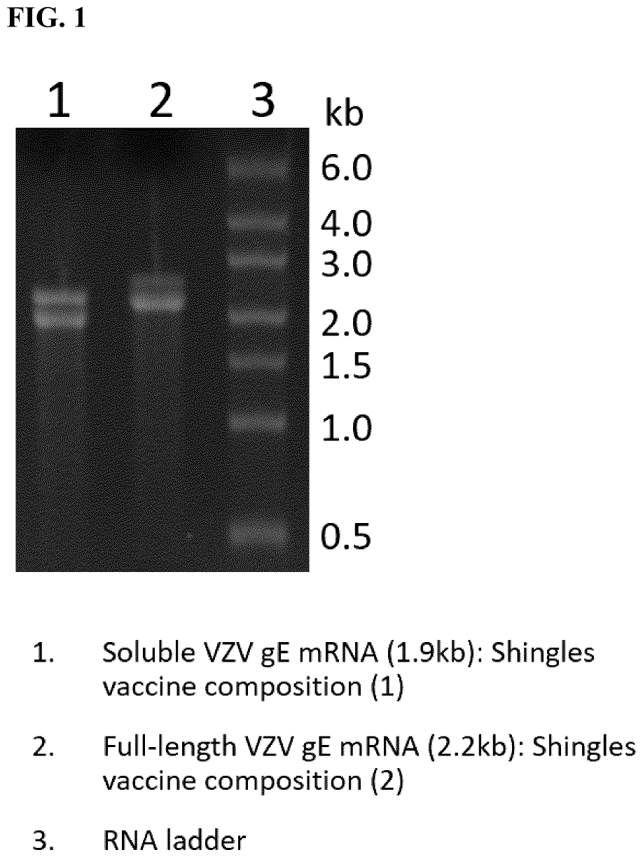

After IVT from DNA templates of VZV gE mRNAs, 100 ng of mRNAs were run on 1% agarose of E-GEL EX in E-Gel Power Snap Electrophoresis Device (ThermoFisher) (one of three independent IVT products). The IVT products of two VZV gE constructs were analyzed by agarose gel, and ˜2 knt long mRNAs for these two mRNAs were detected. See FIG. 1. The IVT was conducted in triplicates.

### Example 3—Transfection

1 ug of mRNAs (synthesized in triplicates) were individually transfected into 293FT cells (Invitrogen) in 12 well plate using Lipofectamine MesseangerMax (Invitrogen), 2 ul at 1:2 ratio according to the manufacturer's protocol. Samples were collected from both media and cells after 24 hours of transfection. Cell lysates were prepared in NP-40 lysis buffer (150 mM sodium chloride/1% NP-40/50 mM Tris pH8.0). As a transfection control, 0.1 ug of EGFP mRNA (L-7601, TriLink) was co-transfected.

### Example 4—Western Blot

Mouse anti-VZV gE monoclonal antibody (#9) was purchased from antibodies.com. Detection of protein was using HRP-conjugated secondary antibodies (Jackson ImmunoResearch, West Grove, PA) and SuperSignal West Pico Plus Chemiluminescent Substrate (Thermo Scientific). EGFP was detected by HRP-conjugated mouse monoclonal antibody (sc-9996, Santa Cruz Biotechnology). The target protein levels were quantitated from the triplicated repeats by analyzing the protein band intensity.

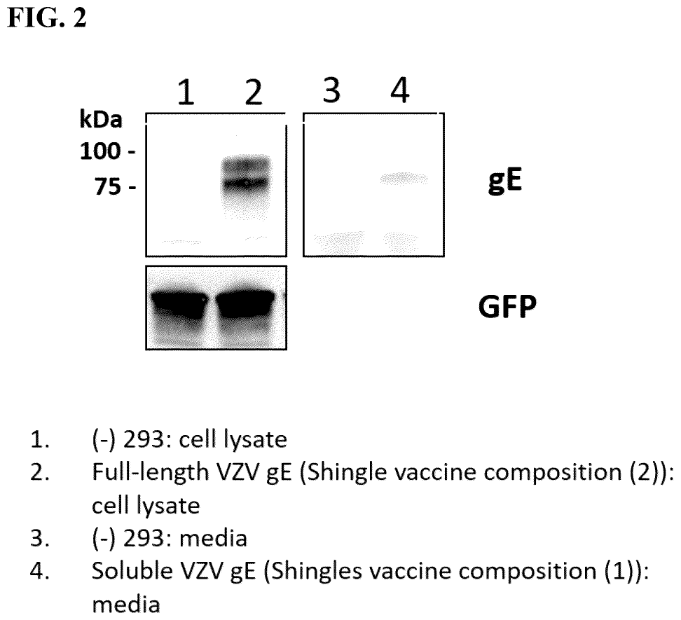

As shown in FIG. 2, the gE protein levels were determined by Western blots. 293 FT cells were individually transfected with 1 ug of the two gE mRNAs. Both cell lysates and culture media were collected at 24 hour post transfection and subjected to Western Blot with gE specific antibody. The GFP mRNA was co-transfected for the normalization, and non-transfected 293 FT cells were used as a negative control (lanes 1 and 3).

Western blots were conducted from collected samples from both media for detecting secreted “soluble” gE proteins and cells for detecting intracellular/un-secreted gE proteins (from three independent repeats). As shown in lane 2 in FIG. 2, the full-length gE protein was detected in cell lysates, but not in media (not shown). Conversely, the soluble gE protein was detected only in the media (lane 4), not in the cell lysates (not shown).

### Example 5—Immunogenicity Study

This study was designed to test the immunogenicity in mice of the shingles vaccine composition of the present disclosure (e.g., shingles vaccine compositions (1) and (2)).

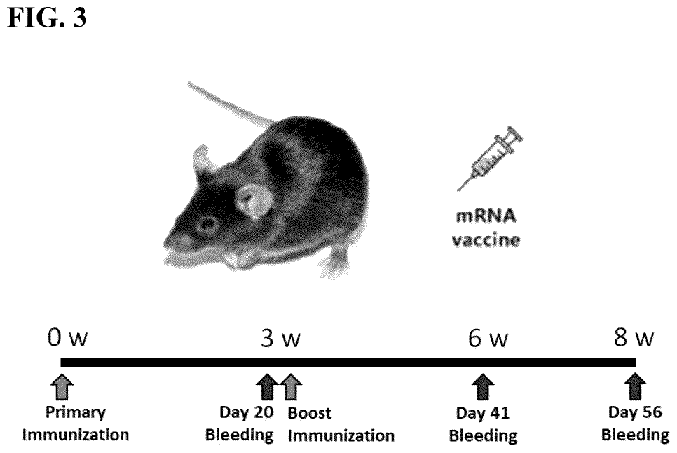

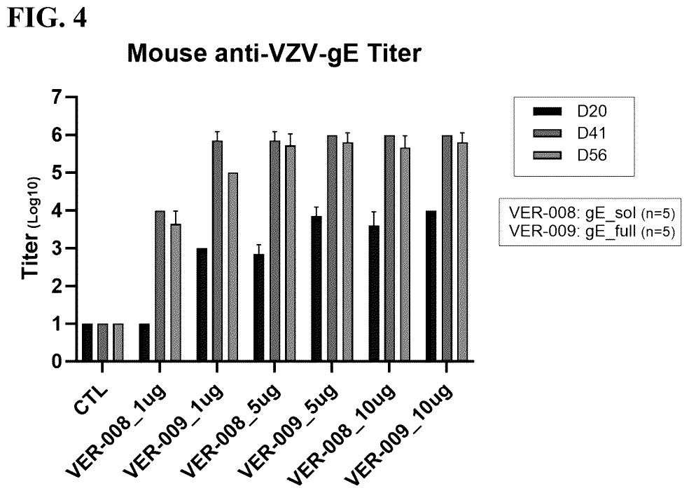

Mice were immunized intramuscularly (IM) with the shingles vaccine composition of the present disclosure (i.e., 1 ug, 5 ug, or 10 ug of mRNA formulation of shingles vaccine compositions (1) or (2) (5 mice per dose). The vaccine composition of the present disclosure is chemically modified or unmodified. A total of two immunizations were given at 3-week intervals (i.e., at weeks 0, and 3), and sera were collected after each immunization until week 8 as shown in FIG. 3 at Day 20, Day 41 and Day 56. Serum antibody titers against VZV gE (soluble), and VZV gE (full-length) were determined by ELISA (AcroBiosystems, RAS-T103). After boost immunization, all the immunized mice showed high IgG titer to VZV gE (D41 and D56 sera) in FIG. 4. The VZV gE-specific IgG titer of shingles vaccine composition (1) (gE full-length) was higher than shingles vaccine composition (2) (gE soluble) at low dose (1 ug) but both compositions show a similar titer at higher doses (5 ug and Mug).

### Example 6—In Vitro Neutralization Study

This study is designed to test the neutralizing capacity of shingles vaccine compositions of the present disclosure (e.g., shingles vaccine compositions (1) to (2)) against VZV infection in cultured human cell line. A patient-isolated VZV strain (human herpesvirus3; ATCC, VR-1367; Ellen strain) was propagated in a human epithelial cell line, ARPE-19 (ATCC, CRL-2302). The infected cells were resuspended in PBS-sucrose-glutamate-serum (PSGC; 5% sucrose, 0.1% L-glutamate, and 10% FBS) buffer and sonicated to release cell-free virus particles with 3 times of 2 min sonication with 15 sec intervals (PC3 Ultrasonic Unit, L&R). The PSGC supernatant containing cell-free VZV was concentrated by combining 3 volume with 1 volume of Lenti-X concentrator (Takara) followed by centrifugation at 1,500×g for 45 min at 4° C. The pellet of VZV was resuspended in 1/50 of initial volume. The viral titer of VZV was determined by infecting ARPE-19 cells with serial dilutions of VZV and measuring gE production using ELISA (AcroBiosystems, RAS-A135).

To assess the mice sera in neutralization of VZV infection, ARPE-19 cells were infected with VZV along with serially diluted mouse sera from the shingles compositions (1) and (2). After 5 days of infection, the amount of gE protein produced from the infected cells was measured in the ELISA assay (AcroBiosystems, RAS-A135) as a neutralizing titer (NT50). FIG. shows the gE full-length sera (VER-009) have higher neutralizing antibodies compared to the gE soluble (VER-008) at all three dose levels. Even at lowest dose (1 ug), the full-length sera were able to elicit strong neutralizing antibodies while the soluble gE needs higher doses for neutralizing capability.

1. A shingles vaccine composition comprising
a messenger ribonucleic acid (mRNA) comprising an open reading frame (ORF) encoding varicella zoster virus (VZV) glycoprotein E (gE).

2. The shingles vaccine composition according to claim 1, wherein the VZV gE has an amino acid sequence of SEQ ID NO: 1.

3. The shingles vaccine composition according to claim 1, wherein the VZV gE has an amino acid sequence of SEQ ID NO: 2.

4. The shingles vaccine composition according to claim 2, wherein the ORF encoding VZV gE has a nucleotide sequence of SEQ ID NO: 3.

5. The shingles vaccine composition according to claim 3, wherein the ORF encoding VZV gE has a nucleotide sequence of SEQ ID NO: 4.

6. The shingles vaccine composition according to claim 2, wherein the mRNA comprising the ORF encoding VZV gE further comprises a 5′ untranslated region (UTR), a 3′ UTR, and a poly (A) tail so as to have the following structure:
5′UTR-ORF encoding VZV gE-3′UTR-poly (A) tail, and
wherein the ORF encoding VZV gE has a nucleotide sequence of SEQ ID NO: 3.

7. The shingles vaccine composition according to claim 3, wherein the mRNA comprising the ORF encoding VZV gE further comprises a 5′ untranslated region (UTR), a 3′ UTR, and a poly (A) tail so as to have the following structure:
5′UTR-ORF encoding VZV gE-3′UTR-poly (A) tail, and
wherein the ORF encoding VZV gE has a nucleotide sequence of SEQ ID NO: 4.

8. The shingles vaccine composition according to claim 6, wherein the poly (A) tail has a length of 50-250 nucleotides.

9. The shingles vaccine composition according to claim 7, wherein the poly (A) tail has a length of 50-250 nucleotides.

10. The shingles vaccine composition according to claim 6, wherein the mRNA having the structure of 5′UTR-ORF encoding VZV gE-3′UTR-poly (A) tail has a nucleotide sequence of SEQ ID NO: 5.

11. The shingles vaccine composition according to claim 7, wherein the mRNA having the structure of 5′UTR-ORF encoding VZV gE-3′UTR-poly (A) tail has a nucleotide sequence of SEQ ID NO: 6.

12. The shingles vaccine composition according to claim 6, wherein the mRNA having the structure of 5′UTR-ORF encoding VZV gE-3′UTR-poly (A) tail has a nucleotide sequence having at least 80% identity to SEQ ID NO: 5.

13. The shingles vaccine composition according to claim 7, wherein the mRNA having the structure of 5′UTR-ORF encoding VZV gE-3′UTR-poly (A) tail has a nucleotide sequence having at least 80% identity to SEQ ID NO: 6.

14. The shingles vaccine composition according to claim 1 further comprising a pharmaceutically acceptable carrier.

15. The shingles vaccine composition according to claim 14, wherein the pharmaceutically acceptable carrier is a lipid nanoparticle encapsulating the mRNA therein.

16. A method of inducing immune response against shingles comprising:
administering an effective amount of the shingles vaccine composition according to claim 1 to a subject in need thereof.

17. A method of inducing immune response against shingles comprising:
administering an effective amount of the shingles vaccine composition according to claim 12 to a subject in need thereof.

18. A method of inducing immune response against shingles comprising:
administering an effective amount of the shingles vaccine composition according to claim 13 to a subject in need thereof.


SEQUENCE LISTING (8 sequences)

SEQ ID NO: 1 - protein, 547 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAA

SEQ ID NO: 2 - protein, 623 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 3 - RNA, 1641 nt, synthetic construct
  source 1..1641: mol_type = other RNA
atgggcaccgtgaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaggagcgtggatttccgccaacggccggtcagccaccggcgactactaaacccaaggaaattacccccgtaaaccccggaacgtcaccacttctacgatatgccgcatggaccggagggcttgcagca

SEQ ID NO: 4 - RNA, 1869 nt, synthetic construct
  source 1..1869: mol_type = other RNA
atgggcaccgtgaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaggagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggactcggaatctacggatacggaagaagagtttggtaacgcgattggagggagtcacgggggttcgagttacacggtgtatatagataagacccgg

SEQ ID NO: 5 - RNA, 1929 nt, synthetic construct
  source 1..1929: mol_type = other RNA
aggccggcactcttctggtccccacagactcagagagaacccgccgccaccatgggcaccgtgaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaggagcgtggatttccgccaacggccggtcagccaccggcgactactaaacccaaggaaattacccccgtaaaccccggaacgtcaccacttctacgatatgccgcatggaccggagggcttgcagcatgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - RNA, 2164 nt, synthetic construct
  source 1..2164: mol_type = other RNA
aggccggcactcttctggtccccacagactcagagagaacccgccgccaccatgggcaccgtgaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaggagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggactcggaatctacggatacggaagaagagtttggtaacgcgattggagggagtcacgggggttcgagttacacggtgtatatagataagacccggtgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - DNA, 4533 bp, synthetic construct
  source 1..4533: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagattaatacgactcactataaggccggcactcttctggtccccacagactcagagagaacccgccgccaccatgggcaccgtgaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaggagcgtggatttccgccaacggccggtcagccaccggcgactactaaacccaaggaaattacccccgtaaaccccggaacgtcaccacttctacgatatgccgcatggaccggagggcttgcagcatgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaagagcatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 8 - DNA, 4768 bp, synthetic construct
  source 1..4768: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagattaatacgactcactataaggccggcactcttctggtccccacagactcagagagaacccgccgccaccatgggcaccgtgaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaggagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggactcggaatctacggatacggaagaagagtttggtaacgcgattggagggagtcacgggggttcgagttacacggtgtatatagataagacccggtgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaagagcatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca